(19) United States
(12) Patent Application Publication
(10) Pub. No.: US 2014/0024112 A1
(43) Pub. Date: Jan. 23, 2014

(54) IN-VIVO BIOREACTOR SYSTEM AND METHOD FOR TISSUE ENGINEERING

(71) Applicant: NORTHWESTERN UNIVERSITY, Evanston, IL (US)

(72) Inventors: Robert D. Galiano, Chicago, IL (US); Thomas A. Mustoe, Evanston, IL (US); Claudia I. Chavez-Munoz, Chicago, IL (US)

(21) Appl. No.: 13/948,957

(22) Filed: Jul. 23, 2013

Related U.S. Application Data
(60) Provisional application No. 61/674,681, filed on Jul. 23, 2012.

(51) Int. Cl. C12M 1/00

(52) U.S. Cl. CPC C12M 29/00
USPC 435/375; 435/289.1; 435/325; 435/395

(57) ABSTRACT

An in-vivo bioreactor system includes a base, a chamber, an access member, an inlet port, an outlet port, and a transparent viewing member. The base includes an internal base cavity. The chamber attaches and detaches from the base. The chamber includes an internal chamber cavity which is in communication with the internal base cavity when the chamber is attached to the base. The access member when disposed in an open position allows access to the internal base cavity or the internal chamber cavity from outside the in-vivo bioreactor system. The inlet port is in communication with the internal base cavity or the internal chamber cavity. The outlet port is in communication with the internal base cavity or the internal chamber cavity. The transparent viewing member allows viewing of the internal base cavity or the internal chamber cavity from outside the in-vivo bioreactor system.

### CROSS REFERENCE TO RELATED APPLICATIONS

The present application claims the benefit of U.S. Provisional Patent Application No. 61/674,681, filed Jul. 23, 2012. The content of this U.S. Provisional Patent Application is hereby incorporated by reference in its entirety.

### STATEMENT REGARDING FEDERALLY SPONSORED RESEARCH OR DEVELOPMENT

This invention was made with government support under Grant No. W81XWH-11-1-0839 awarded by the US Army Medical Research and Materiel Command (Army/MRMC). The government has certain rights in the invention.

### FIELD OF THE DISCLOSURE

The disclosure relates to a tissue regeneration, growth, and/or treatment device comprising an in-vivo bioreactor systems and methods for tissue engineering within a living creature.

### BACKGROUND OF THE DISCLOSURE

There is a great need for the ability to replace organs and tissues in living creatures due to injury and disease. Unfortunately replacement organs, and tissue are not readily available, and immunosuppression drugs required for transplants have considerable side-effects. Scientists have developed external bioreactor systems where cells, tissue, or organs can grow. However, it has proven difficult to transplant these cells, tissues, or organs into living creatures due to the lack of vasculature. Secondly, there currently exist a number of biological wound care products that have practicality and feasibility issues. These include accellular and cellular dermal matrices, growth factors, and cell transplant technology. As a result there is a large need for improvement of existing technologies or methods to improve the outcomes of their use. To overcome these issues, scientists have begun working on in-vivo bioreactor systems in which the bioreactor system is implanted into the living creature. There have been many issues with these in-vivo bioreactor systems such as: the difficulty in not being able to visualize the growing tissues, or organs; the difficulty in determining properties of the growing tissue, or organs and its surrounding environment; the difficulty in delivering fluids, such as mediums or other agents, to the growing/regenerating tissue or organ to help it grow; the difficulty in applying stimuli to the growing tissue or organ to help it grow; the difficulty in accessing the growing tissue or organ; the inability to vary, adapt, or change-out the in-vivo bioreactor system to obtain varying functions for the growing tissue or organ after the in-vivo bioreactor system is implanted; the difficulty in adding sequential cell populations in different layers/configurations to generate tissue or an organ; and additional issues.

There is a need for an in-vivo bioreactor system which will resolve one or more of the issues associated with the current systems.

### SUMMARY OF THE DISCLOSURE

In one embodiment, an in-vivo bioreactor system includes a base, a chamber, an access member, an inlet port, an outlet port, and a transparent viewing member. The base includes an internal base cavity. The chamber attaches and detaches from the base. The chamber includes an internal chamber cavity which is in communication with the internal base cavity when the chamber is attached to the base. The access member when disposed in an open position allows access to the internal base cavity or the internal chamber cavity from outside the in-vivo bioreactor system. The inlet port is in communication with the internal base cavity or the internal chamber cavity. The outlet port is in communication with the internal base cavity or the internal chamber cavity. The transparent viewing member allows viewing of the internal base cavity or the internal chamber cavity from outside the in-vivo bioreactor system.

In another embodiment, an in-vivo bioreactor system attached to a living creature includes a base, a chamber, an access member, an inlet port, an outlet port, and a transparent viewing member. The base includes an internal base cavity. The base is attached to the living creature with a portion of the base disposed underneath a dermis of the living creature. The chamber is attached to the base. The chamber includes an internal chamber cavity which is in communication with the internal base cavity. The access member is attached to the chamber. When the access member is disposed in an open position access is provided to the internal base cavity or the internal chamber cavity from outside skin of the living creature. The inlet port is in communication with the internal base cavity or the internal chamber cavity. The outlet port is in communication with the internal base cavity or the internal chamber cavity. The transparent viewing member allows viewing of the internal base cavity or the internal chamber cavity from outside the skin of the living creature.

In an additional embodiment, a method of using an in-vivo bioreactor system is disclosed. In one step, a base of an in-vivo bioreactor system is located to be at least partially disposed below a dermis of the living creature. In another step, a chamber of the in-vivo bioreactor system is attached to the base so that an internal chamber cavity of the chamber is in communication with an internal base cavity of the base. In an additional step, a medium is flowed through an inlet port of the in-vivo bioreactor system into the internal base cavity or the internal chamber cavity while the chamber is attached to the base attached to the living creature. In still another step, a tissue or an organ is grown within the internal base cavity or the internal chamber cavity. In yet another step, the growing tissue or the growing organ disposed within the internal base cavity or the internal chamber cavity is viewed through a transparent viewing member of the in-vivo bioreactor system

These and other features, aspects and advantages of the disclosure will become better understood with reference to the following drawings, description and claims.

### BRIEF DESCRIPTION OF THE DRAWINGS

FIG. 1 illustrates a disassembled perspective view of one embodiment of an in-vivo bioreactor system comprising a base, a chamber, and an access member;

FIG. 2 illustrates a perspective view of the base of the in-vivo bioreactor system of FIG. 1;

FIG. 3 illustrates a perspective view of the chamber of the in-vivo bioreactor system of FIG. 1;

FIG. 4 illustrates a perspective view of the access member of the in-vivo bioreactor system of FIG. 1;

FIG. 5 illustrates a partially assembled perspective view of the in-vivo bioreactor system of FIG. 1 with the base attached to a dermis of a living creature and the access member detached from the chamber;

FIG. 6 illustrates a cross-section view through line 6-6 of FIG. 5;

FIG. 7 illustrates a fully assembled perspective view of the in-vivo bioreactor system of FIG. 1 attached to the dermis of the living creature with the access member attached to the chamber in a closed position and external systems connected to the bioreactor system;

FIG. 8 illustrates a cross-section view through line 8-8 of FIG. 7;

FIG. 9 illustrates a perspective view of a plurality of representative modular, adaptable, varying chambers which may each separately attach and detach from the base and access member of the in-vivo bioreactor system of FIG. 1 to achieve varying functions while creating and growing a tissue, or an organ in a living creature; and

FIG. 10 is a flowchart illustrating one embodiment of a method of using an in-vivo bioreactor system.

### DETAILED DESCRIPTION OF THE DISCLOSURE

The following detailed description is of the best currently contemplated modes of carrying out the disclosure. The description is not to be taken in a limiting sense, but is made merely for the purpose of illustrating the general principles of the disclosure, since the scope of the disclosure is best defined by the appended claims.

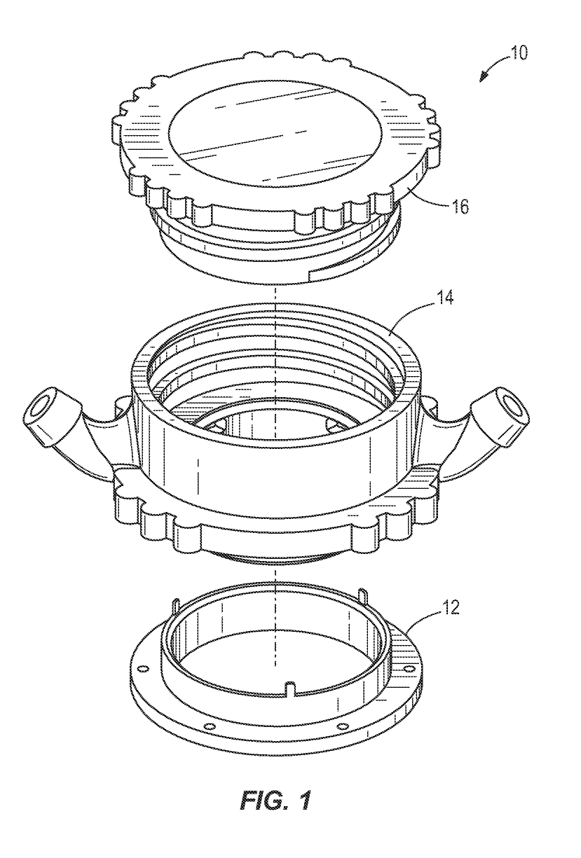

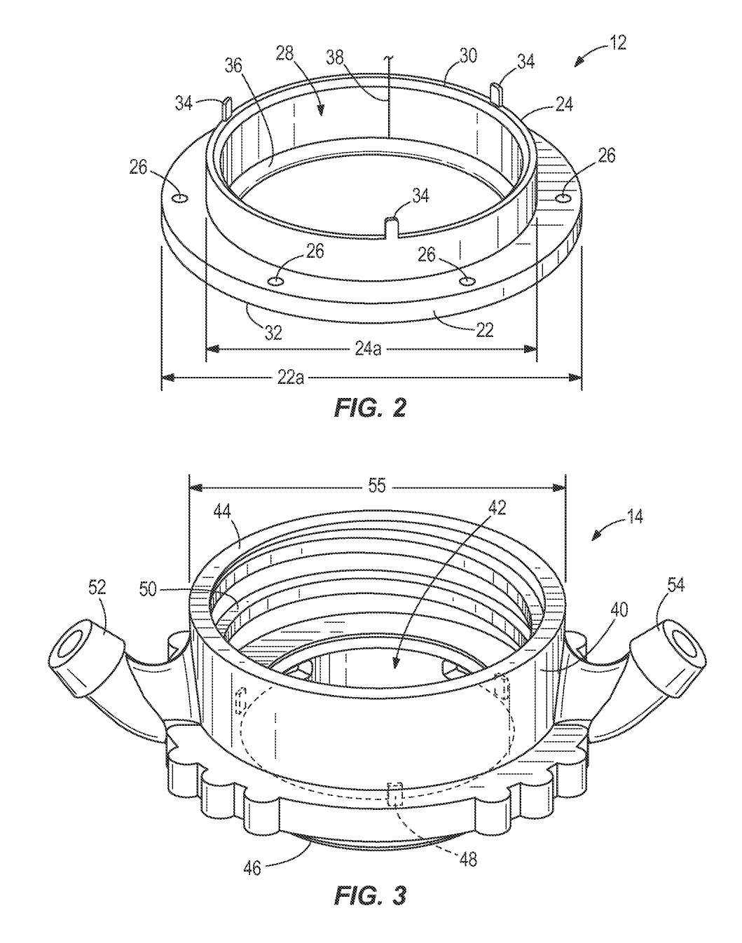

FIG. 1 illustrates a disassembled perspective view of one embodiment of an in-vivo bioreactor system 10 comprising a base 12, a chamber 14, and an access member 16. FIG. 2 illustrates a perspective view of the base 12 of the in-vivo bioreactor system 10 of FIG. 1. As shown in FIG. 2, the base 12 comprises attached cylindrical rings 22 and 24, with cylindrical ring 22 having a larger diameter 22a than the diameter 24a of cylindrical ring 24, holes 26 extending around a perimeter of cylindrical ring 22, an internal base cavity 28 extending between opposed surfaces 30 and 32, base attachment members 34, and an adaptable stimuli member 36. The base attachment members 34 comprise tabs but in other embodiments may comprise any number or type of attachment members for attaching and detaching the base 12 to the chamber 14 (of FIG. 1) such as fasteners, male members, female members, threaded members, holes, or other types of attachment members. The stimuli member 36 may be used to apply stimulation such as mechanical, electrical, or chemical within or adjacent to the internal base cavity 28. The stimuli member 36 may apply a tension or compression stress or strain, electrical impulses, or chemical stimulation. The stimuli member 36 may comprise a circular, biocompatible, silicone membrane which is attached to a member 38. The member may comprise a motor for mechanical stimulation, a wire or an array of wires for electrical stimulation, micro or nanofibers, micro/nano spheres for a chemical network or chemical delivery, or other type of member. In other embodiments, the stimuli member 36 may be disposed within or adjacent to an internal cavity of the chamber 14 (of FIG. 1), or may vary in material, shape, size, type, or function. In still other embodiments, the stimuli member 36 may be used to assist with tissue printing, stretching, to provide morphogenic cues, to provide stretch/strain or other mechanoresponsive stimuli, for flow or pressure stimuli or electrical stimuli, or to provide other stimuli to influence the growth of tissue.

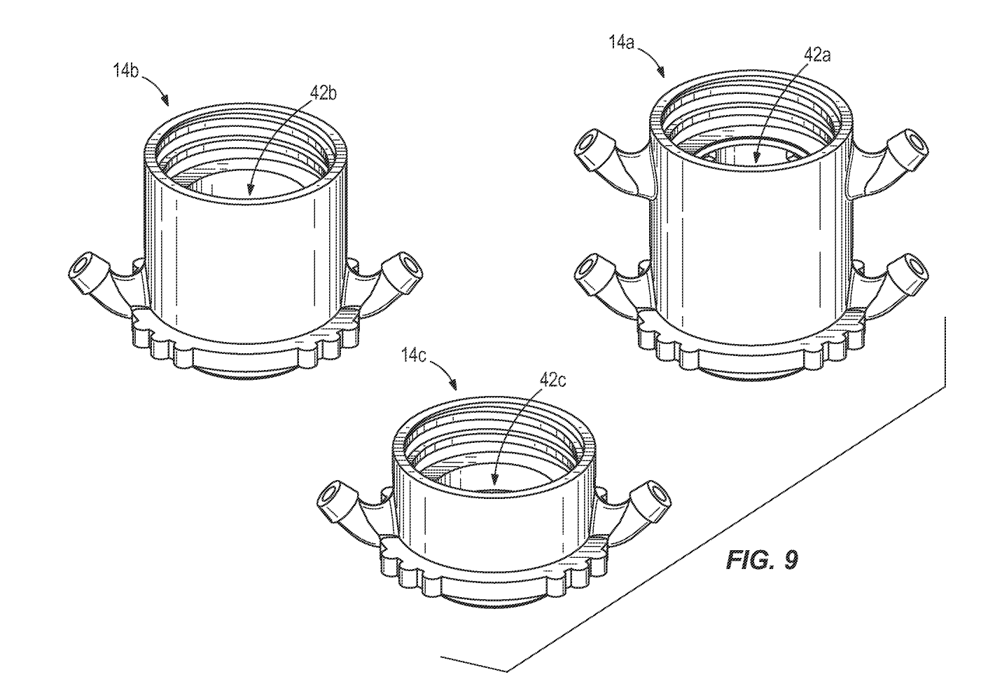

The base 12 is made of a biocompatible material such as but not limited to: silicone, thermoplastic elastomers, polypropylene, titanium watershed XC 11122, or other types of biocompatible materials. In other embodiments, the base 12 may be of varying shapes, sizes, type, or materials. The base 12 is designed to not only anchor the chamber 14 of FIG. 1 to a living creature, but also may exclude dermis of the living creature so as to prevent healing over the device unless it is necessary. In another embodiment, the base 12 may not exclude dermis of the living creature. As discussed below, the base 12 is designed to attach to different types of varying, adaptable modular chambers 14a, 14b, and 14c (shown in FIG. 9) according to the needs of the experiment or clinical scenario. These modular chambers 14a, 14b, and 14c may include, but are not limited to, perfusion systems or microfluidics, sensors, modules that produce intermittent stress or strain of the tissue construct, modules that deliver negative pressure or electric current or shockwaves, modules that are laminated to create a multi-layered tissue or organ, or other types of modular chambers.

FIG. 3 illustrates a perspective view of the chamber 14 of the in-vivo bioreactor system 10 of FIG. 1. As shown in FIG. 3, the chamber 14 comprises a cylindrical ring 40, an internal chamber cavity 42 extending between opposed surfaces 44 and 46, chamber attachment members 48 and 50, and inlet and outlet ports 52 and 54. The inlet and outlet ports 52 and 54 are in communication with the internal chamber cavity 42. In other embodiments, the chamber 14 may comprise a varying number of ports in varying positions or no ports at all. In still other embodiments, one or more inlet or outlet ports may be disposed in the base 12 or the access member 16 of FIG. 1. The chamber attachment members 48 comprise holes but in other embodiments may comprise any number or type of attachment members for attaching and detaching the chamber 14 to the base 12 (shown in FIG. 1) such as threaded members, fasteners, tabs, female members, male members, or other types of attachment members. The chamber attachment member 50 comprises a threaded hole but in other embodiments may comprise any number or type of attachment member for attaching and detaching the chamber 14 to the access member 16 (shown in FIG. 1) such as a female member, a fastener, a tab, a male member, or another type of attachment member.

The chamber 14 is made of biocompatible material such as but not limited to: silicone, thermoplastic elastomers, polypropylene, titanium, watershed XC 11122, or other types of biocompatible materials. An inner diameter 55 of the chamber 14 accommodates different matrices, sensors, and nutrients to allow the chamber 14 to be used for numerous applications. In other embodiments, the chamber 14 may be of varying shapes, sizes, type, or materials. The chamber 14 is implantable and designed to be in contact with tissues and blood vessels to provide the vascular network essential to grow complex, 3-dimensional tissues or organs. The chamber 14 is designed to contain matrices of any biocompatible type, or engineered or micropatterned scaffolds (including decellularized tissues or imprinted templates for tissue growth). The chamber 14 is flexible and can work with any biocompatible matrix or engineered system that can be implanted in-vivo.

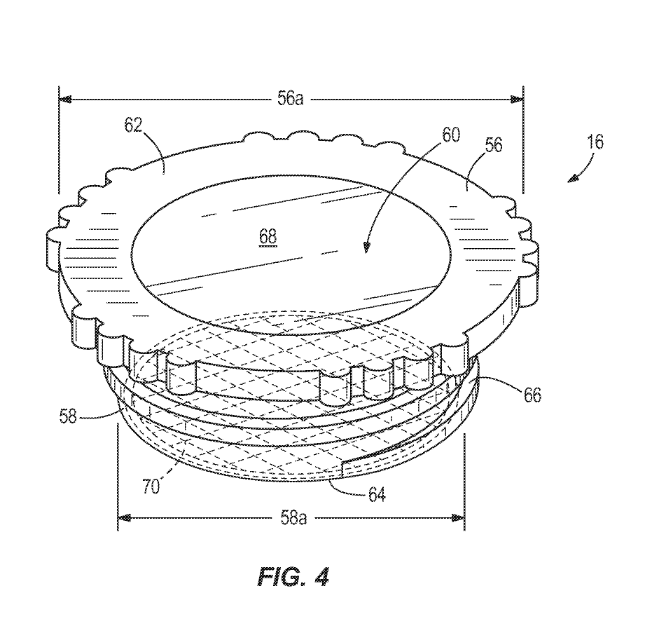

FIG. 4 illustrates a perspective view of the access member 16 of the in-vivo bioreactor system 10 of FIG. 1. As shown in FIG. 4, the access member 16 comprises attached cylindrical rings 56 and 58 with cylindrical ring 56 having a larger diameter 56a than the diameter 58a of cylindrical ring 58, an internal access member cavity 60 extending between opposed surfaces 62 and 64, access attachment member 66, transparent viewing member 68, and sensor 70. The access member 16 may comprise a cap. The access attachment member 66 comprises a threaded male member but in other embodiments may comprise any type of attachment member for attaching and detaching the access member 16 to the chamber 14 (shown in FIG. 1) such as a fastener, a tab, a threaded female member, or another type of attachment member. The transparent viewing member 68 may be made of any type of transparent material. The sensor 70 may comprise any number or type of sensor such as a temperature sensor, a pH sensor, an oxygen sensor, a flow sensor, a glucose sensor, a protein sensor, a biological product sensor (such as but not limited to: cytokines, growth factors, metabolites, etc.), or another type of sensor. In other embodiments, the transparent viewing member 68 or the sensor 70 may be disposed in the chamber 14 or the base 12 of FIG. 1.

The access member 16 is made of biocompatible materials such as but not limited to: silicone, thermoplastic elastomers, polypropylene, titanium, watershed XC 11122, or other types of biocompatible materials. In other embodiments, the access member 16 may be of varying shapes, sizes, type, or materials. As discussed more thoroughly below, the access member 16 is configured to, when attached to the chamber 14 of FIG. 1 in an in-vivo bioreactor system 10 which has been implanted in a living creature, exclude the external environment, allow for the delivery of, but not limited to fluids, cells, nutrients, or other mediums, through ports or valves, allow for the visualization of growing tissue within the in-vivo bioreactor system 10, or provide varying other functions.

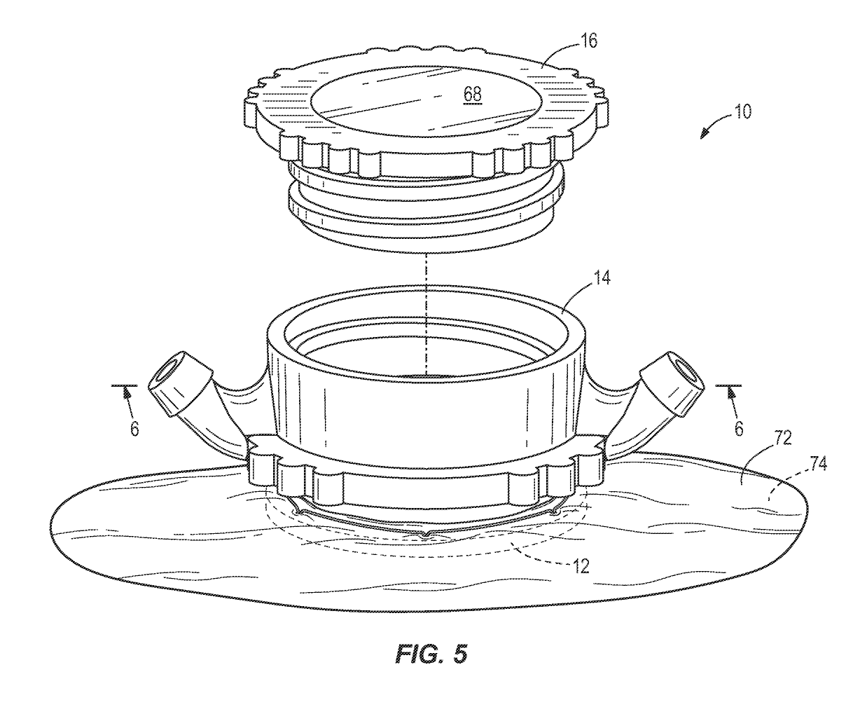

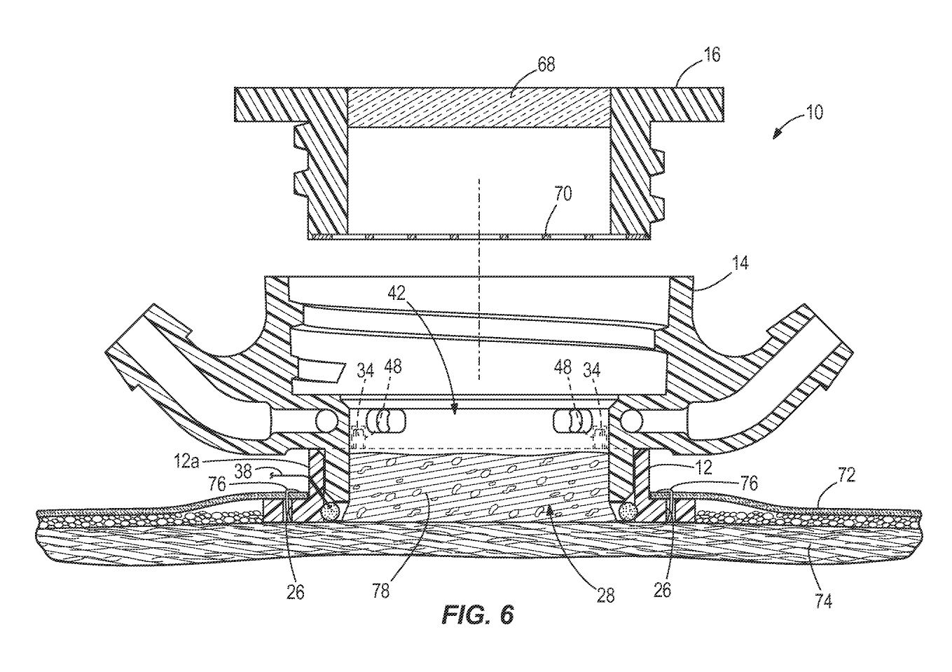

FIG. 5 illustrates a partially assembled perspective view of the in-vivo bioreactor system 10 of FIG. 1 with the base 12 attached to a dermis 72 of a living creature 74 and the access member 16 detached from the chamber 14. The living creature 74 may comprise any type of living creature such as a human, an animal, or another type of living creature. FIG. 6 illustrates a cross-section view through line 6-6 of FIG. 5. As shown collectively in FIGS. 5 and 6, the base 12 is attached to the living creature 74 with a portion 12a of the base 12 disposed underneath the dermis 72 of the living creature 74. The base 12 is attached to the living creature 74 with securement members 76 extending through the holes 26 of the base 12 into the dermis 72 of the living creature 74. The securement members 76 comprise stiches but in other embodiments may comprise any number or type of securement member for attaching the base 12 to the living creature 74 such as staples, fastening members, or other types of securement members. The base attachment members 34 of the base 12 are attached to the chamber attachment members 48 of the chamber 14 detachably securing the base 12 to the chamber 14. The access member 16 is detached from the chamber 14 in an open position allowing access to the internal base cavity 28 and the internal chamber cavity 42 from outside skin of the dermis 72 and from outside the in-vivo bioreactor system 10. A substance 78 comprising a biocompatible engineered system, a biocompatible matrix, or a biocompatible scaffold has been inserted into the internal base cavity 28 and the internal chamber cavity 42 through the internal chamber cavity 42 which is possible because the access member 16 is detached from the chamber 14.

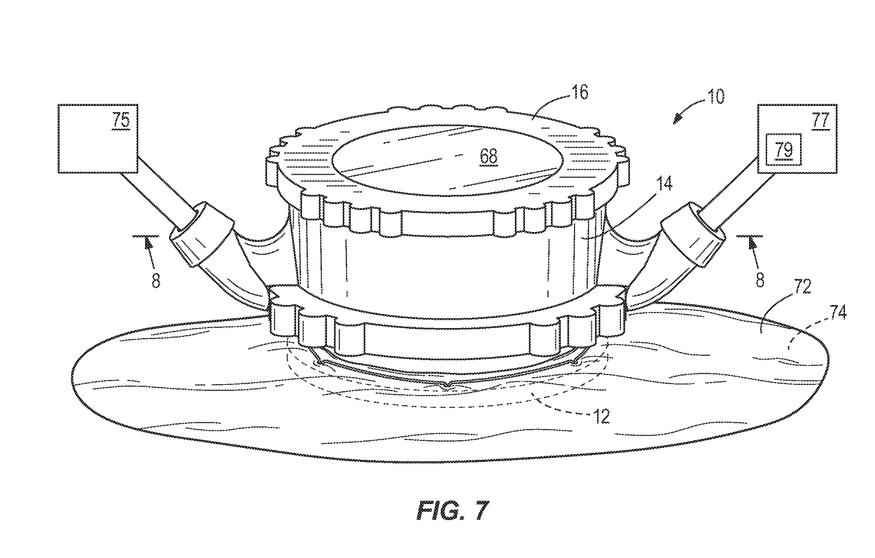

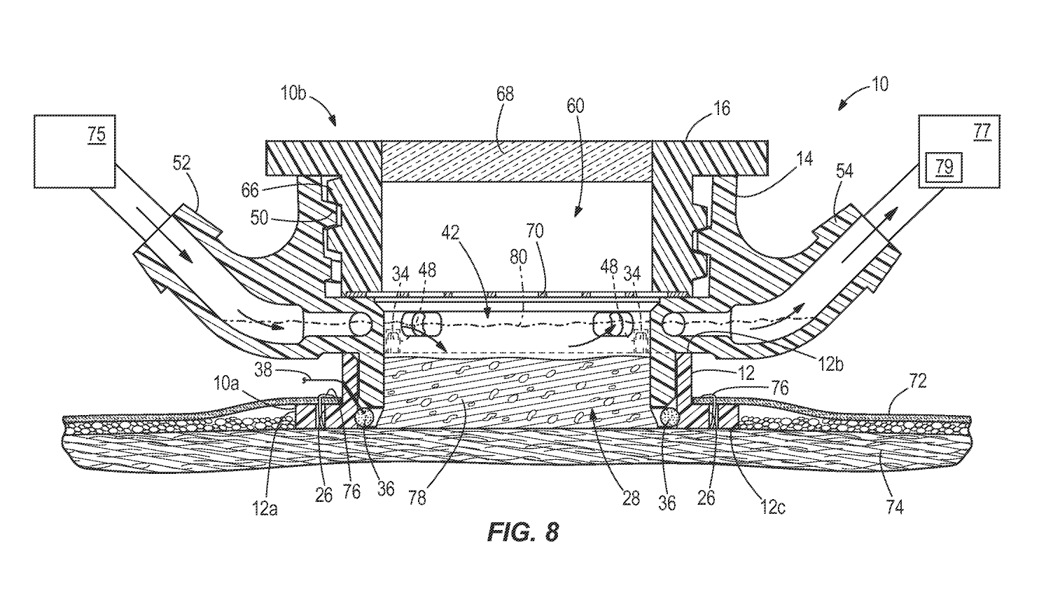

FIG. 7 illustrates a fully assembled perspective view of the in-vivo bioreactor system 10 of FIG. 1 attached to the dermis 72 of the living creature 74 with the access member 16 attached to the chamber 14 in a closed position and external systems 75 and 77 connected to the bioreactor system 10. FIG. 8 illustrates a cross-section view through line 8-8 of FIG. 7. The access member 16 was attached to the chamber 14 into its closed position by attaching the access attachment member 66 of the access member 16 to the chamber attachment member 50 of the chamber 14 securing the access member 16 to the chamber 14 with the substance 78 disposed within the internal base cavity 28 and the internal chamber cavity 42. With the base 12 attached to the chamber 14 and the chamber 14 attached to the access member 16, the internal base cavity 28, the internal chamber cavity 42, and the internal access member cavity 60 are all in communication with one another and are also all in communication with the inlet and outlet ports 52 and 54 of the chamber 14. The inlet and outlet ports 52 and 54 are in communication with respective external systems 75 and 77.

In this attached configuration, the transparent viewing member 68 allows viewing of the internal base cavity 28, the internal chamber cavity 42, and the internal access member cavity 60 from outside the in-vivo bioreactor system 10 and from outside the skin of the dermis 72 of the living creature 74. This allows viewing of the substance 78 disposed within the internal base cavity 28 and the internal chamber cavity 42 so that it can be determined how well the substance 78 is growing to create a tissue or an organ. In this attached configuration access to the internal base cavity 28, the internal chamber cavity 42, and the internal access member cavity 60 is closed from outside skin of the dermis 72 and is closed from a side 10a and above 10b the in-vivo bioreactor system 10 with the exception of through the inlet and outlet ports 52 and 54. A medium 80 has been flowed from external system 75 through the inlet port 52, through the internal chamber cavity 42, and into internal base cavity 28. The external system 75 comprises a perfusion system (which may include a perfusion pump) for delivering and regulating the medium 80 to the inlet port 52. The medium 80 comprises a growth factor, a protein, a nutrient, a cell, a liquid scaffold, a medication, a treatment, or another type of medium for assisting the substance 78 to grow to create a tissue, or an organ within the living creature 74. The outlet port 54 may be used to suction out the medium 80 from within the internal base cavity 28 and the internal chamber cavity 42 to external system 77. External system 77 comprises an external monitoring device which monitors and analyzes the medium 80 or tissue or organ material as it's suctioned out using sensors 79 in order to monitor in real-time the tissue or organ and the tissue growth/regeneration. The sensors 79 may detect oxygen, pH, temperature, glucose, protein, metabolite, flow, biological product, or other items. Collectively, the inlet and outlet ports 52 and 54, using the external systems 75 and 77, may be used to deliver and regulate mediums 80, to provide suction for aspiration or as a vacuum, to monitor and analyze the tissue or organ and the tissue growth/regeneration, or for other purposes. Through inlet and outlet ports 52 and 54 any number of therapeutics, reagents, nutrients, growth factors, liquid scaffolds, medications, treatments, or other mediums may be administered in a manual or automated fashion (controlled perfusion rate) to aid tissue or organ healing and tissue growth/regeneration. In other embodiments, any number of external systems of varying type may be connected to the inlet and/or outlet ports 52 and 54 of the bioreactor system 10.

In this attached configuration the sensor 70 disposed within or adjacent to the internal base cavity 28, the internal chamber cavity 42, or the internal access member cavity 60 is disposed against or adjacent to the substance 78. In such manner, the sensor 70 may be used to take various sensor measurements to determine the conditions in which the substance 78 is growing, to determine how well the substance 78 is growing, or to take other sensor measurements to make other determinations. In this attached configuration, the stimuli member 36 disposed within or adjacent to the internal base cavity 28, the internal chamber cavity 42, or the internal access member cavity 60 is disposed against or adjacent to the substance 78 applying stimulation, powered by member 38 (only partially shown for ease of illustration), to assist in creating and growing a tissue or organ.

FIG. 9 illustrates a perspective view of a plurality of representative modular, adaptable, varying chambers 14a, 14b, and 14c which may each separately attach and detach from the base 12 and access member 16 of the in-vivo bioreactor system of FIG. 1 to achieve varying functions while creating and growing a tissue or an organ in a living creature using the in-vivo bioreactor system 10 of FIG. 1. The varying functions which may be achieved using the modular, adaptable, varying chambers 14a, 14b, and 14c may relate to growing or obtaining varying properties, growth levels, growth stages, or results for the created and growing tissue, or the created and growing organ. For instance, in one embodiment sequentially chamber 14a may be used to grow an organ, chamber 14b may be used to grow a muscle, and chamber 14c may be used to grow skin. In another embodiment, sequentially chamber 14a may be used to grow an organ, a muscle, or skin to a certain point, chamber 14b may be used to grow the organ, the muscle, or the skin to another point, and chamber 14c may be used to grow the organ, the muscle, or the skin to a final point. In other embodiments, a varying number and type of modular and adaptable chambers may be used in any order to achieve any desired function or result.

Each of the varying chambers 14a, 14b, and 14c may have an internal chamber cavity 42a, 42b, and 42c which will be in communication with the internal base cavity 28 (shown in FIG. 2) and the internal access member cavity 60 (shown in FIG. 4) when the varying chambers 14a, 14b, and 14c are separately attached to the base 12 and the access member 16 of FIG. 1. The plurality of varying chambers 14a, 14b, and 14c may vary relative to one another in at least one of size, structure, number or type of inlet or outlet ports, number or type of sensors, number or type of stimuli members, or may vary in one or more other structural ways. The modular chambers 14a, 14b, and 14c may include, but are not limited to, perfusion systems or microfluidics, sensors, modules that produce intermittent stress or strain of the tissue construct, electrical stimulation, modules that deliver negative pressure or shockwaves, or other forms of energy modules that are laminated to create multi-layered tissue, or other types of modular chambers. The external systems 75 and 77 of FIG. 7 may be connected to any of the varying chambers 14a, 14b, and 14c. In other embodiments, a varying number of external systems of varying type may be connected to any of the varying chambers 14a, 14b, and 14c.

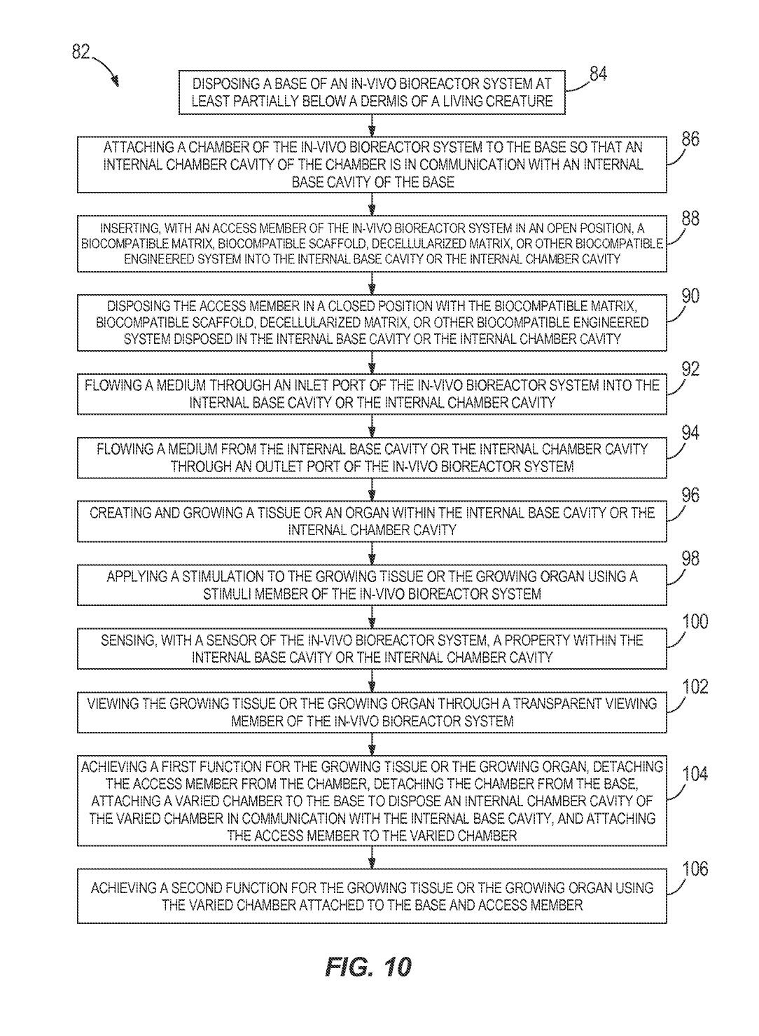

FIG. 10 is a flowchart illustrating one embodiment of a method 82 of using an in-vivo bioreactor system. In step 84, a base of an in-vivo bioreactor system is at least partially disposed below a dermis of a living creature. The base may be attached to the living creature using securement members attached between the base and a dermis of the living creature. In other embodiments, the base may be attached to the living creature in varying ways. In step 86, a chamber of the in-vivo bioreactor system is attached to the base so that an internal chamber cavity of the chamber is in communication with an internal base cavity of the base. In step 88, a biocompatible matrix, a biocompatible scaffold, a decellularized matrix, or other biocompatible engineered system is inserted, with an access member of the in-vivo bioreactor system in an open position, into the internal base cavity of the base or the internal chamber cavity of the chamber. In step 90, the access member is disposed in a closed position with the biocompatible matrix, the biocompatible scaffold, the decellularized matrix, or other biocompatible engineered system disposed in the internal base cavity or the internal chamber cavity.

In step 92, a medium is flowed through an inlet port of the in-vivo bioreactor system into the internal base cavity or the internal chamber cavity while the chamber is attached to the base attached to the living creature. Step 92 may comprise an external system (such as an external pump) delivering the medium to the inlet port. In step 94, the medium is flowed from the internal base cavity or the internal chamber through an outlet port of the in-vivo bioreactor system. The medium comprises a growth factor, protein, nutrient, a liquid scaffold, a medication, a treatment, or another medium, and may or may not contain cells, or another medium for assisting the biocompatible matrix, the biocompatible scaffold, the decellularized matrix, or the other biocompatible engineered system disposed within the internal base cavity or the internal chamber to be created and grow into a tissue or an organ. In step 96, a tissue or an organ is created and grown within the internal base cavity or the internal chamber cavity from the biocompatible matrix, the biocompatible scaffold, the decellularized matrix, or the other biocompatible engineered system. In step 98, stimulation is applied to the growing tissue, or the growing organ disposed within the internal base cavity or the internal chamber cavity using a stimuli member of the in-vivo bioreactor system. The stimulation being applied may comprise a tension or compression stress or strain, electrical stimulation, or another type of stimulation.

In step 100, a property within the internal base cavity or the internal chamber cavity is sensed using a sensor or an array of sensors of the in-vivo bioreactor system. The sensed property may comprise a temperature, a pH, an oxygen level, a flow, a protein, a glucose, a biological product such as cytokines, growth factors, metabolites, or another property which may be useful to know in growing the biocompatible matrix, the biocompatible scaffold, the decellularized matrix, or the other biocompatible engineered system into a tissue or an organ. In another embodiment, step 100 may comprise an external system sensing the property by testing the medium or a material of the tissue or organ after it exits (such as through suction) the in-vivo bioreactor system. In step 102, the growing tissue, or the growing organ disposed within the internal base cavity or the internal chamber cavity is viewed through a transparent viewing member of the in-vivo bioreactor system.

In step 104, after the chamber has been used to achieve a first function for the growing tissue or the growing organ, the access member is detached from the chamber, the chamber is detached from the base, a varied chamber is attached to the base to dispose an internal chamber cavity of the varied chamber in communication with the internal base cavity, and the access member is attached to the varied chamber. The varied chamber may vary from the original chamber in size, structure, number or type of inlet or outlet ports, number or type of sensors or sensor arrays, or may vary in another way. In step 106, the varied chamber attached to both the base and the access member is used to achieve a second function for the growing tissue, or the growing organ. The first and second functions of the varied chambers may comprise growing or obtaining varied properties, growth levels, growth stages, or results for the created and growing tissue, or the created and growing organ. In other embodiments, the first and second functions may vary. One or more external systems may be connected to the varied chambers to achieve and/or monitor the first and second functions.

One or more embodiments of the disclosure may reduce one or more problems associated with one or more of the existing in-vivo bioreactor systems and methods. For instance, the placement of the in-vivo bioreactor system within a living creature provides the vascular network necessary for tissues and complex tissues, such as organs to be engineered in-situ ad long-term. The modularity of the in-vivo bioreactor system, including the ports, allows for mediums to be intermittently or continuously delivered such as but not limited to growth factors, nutrients, therapeutics, cells, chemicals, liquid scaffolds, medications, treatments, or matrices in order to both enhance tissue growth as well as direct and orchestrate cellular depositions/organization. The adaptability of the chamber extends its utility beyond solely the delivery of mediums, in that it also provides the opportunity of incorporating sensors in isolation or in arrays that can measure variables including but not limited to oxygen, flow, pH, temperature, glucose, protein, and can provide biological products like cytokines, growth factors, metabolites, or other substances in order to monitor and track any changes in the tissue regeneration process. Moreover, one or more external systems may be connected to the in-vivo bioreactor system to deliver, monitor, analyze, and regulate the medium or a material of the tissue or organ to analyze, control, and regulate the tissue or organ and the tissue growth/regeneration.

The adaptable nature of the device further permits incorporation of a variety of matrices and growth of tissue in a controllable manner, including but not limited to tissue printing, stretching, and other interventions that can influence the growth of tissue in a manner responsive to a wide variety of stimuli, such as mechanical, electrical, chemical, or other types of stimuli. These stimuli can be built into different varying modular chambers, and may range from morphogenic cues, to stretch/strain and other mechanoresponsive stimuli, to flow or negative pressure stimuli, or other stimuli. The modular, adaptable chamber permits the sequential addition of different cell populations in different layers/configurations to generate biomimetic tissue and organs. Different modules may be used according to the discrete needs of the growing tissue. The transparent viewing member and the removable access member allow for access to and viewing of the growing tissue, or organ. The in-vivo bioreactor systems and methods of the disclosure may be used for tissue engineering, angiogenesis, lymphangiogenesis, cellular proliferation, and for other uses.

It should be understood, of course, that the foregoing relates to exemplary embodiments of the disclosure and that modifications may be made without departing from the spirit and scope of the disclosure as set forth in the following claims.

We claim:

1. An in-vivo bioreactor system comprising:
a base having an internal base cavity;
a chamber which attaches and detaches from the base, the chamber having an internal chamber cavity which is in communication with the internal base cavity when the chamber is attached to the base;
an access member which when disposed in an open position allows access to the internal base cavity or the internal chamber cavity from outside the in-vivo bioreactor system;
an inlet port in communication with the internal base cavity or the internal chamber cavity;
an outlet port in communication with the internal base cavity or the internal chamber cavity; and
a transparent viewing member which allows viewing of the internal base cavity or the internal chamber cavity from outside the in-vivo bioreactor system.

2. The in-vivo bioreactor system of claim 1 wherein the access member attaches and detaches from the chamber.

3. The in-vivo bioreactor system of claim 2 wherein the access member comprises a cap.

4. The in-vivo bioreactor system of claim 3 wherein the cap comprises the transparent viewing member.

5. The in-vivo bioreactor system of claim 1 wherein when the access member is disposed in a closed position, access to the internal base cavity or the internal chamber cavity is closed from above and to a side of the in-vivo bioreactor system with the exception of through the inlet and outlet ports.

6. The in-vivo bioreactor system of claim 1 wherein the inlet and outlet ports are disposed in the chamber.

7. The in-vivo bioreactor system of claim 1 further comprising at least one sensor disposed within or adjacent to the internal base cavity or the internal chamber cavity.

8. The in-vivo bioreactor system of claim 7 wherein the at least one sensor comprises at least one of a temperature sensor, a pH sensor, an oxygen sensor, a flow sensor, a glucose sensor, a protein sensor, or a biological product sensor.

9. The in-vivo bioreactor system of claim 1 further comprising a stimuli member disposed within or adjacent to the internal base cavity or the internal chamber cavity for applying stimulation.

10. The in-vivo bioreactor system of claim 9 wherein the stimuli member applies a tension or compression stress or strain.

11. The in-vivo bioreactor system of claim 9 wherein the stimuli member comprises a membrane which is attached to an electrical wire or other member for moving the membrane.

12. The in-vivo bioreactor system of claim 1 wherein the in-vivo bioreactor system is made of a biocompatible material.

13. The in-vivo bioreactor system of claim 1 further comprising a plurality of varying chambers which each separately attach and detach from the base for achieving varying functions, each of the varying chambers having an internal chamber cavity which is in communication with the internal base cavity when the chamber is attached to the base.

14. The in-vivo bioreactor system of claim 13 wherein the plurality of varying chambers vary in at least one of size, structure, number or type of inlet or outlet ports, or number or type of sensors.

15. The in-vivo bioreactor system of claim 13 wherein the access member attaches and detaches to each of the plurality of varying chambers.

16. The in-vivo bioreactor system of claim 1 further comprising at least one external system which is configured to deliver a medium to the inlet port, or to monitor or analyze a property of the medium or material of a tissue or organ after it exits the outlet port.

17. The in-vivo bioreactor system of claim 16 wherein the at least one external system comprises a perfusion system, a temperature sensor, a pH sensor, an oxygen sensor, a flow sensor, a glucose sensor, a protein sensor, or a biological product sensor.

18. An in-vivo bioreactor system attached to a living creature comprising:
a base having an internal base cavity, the base attached to the living creature with at least a portion of the base disposed underneath a dermis of the living creature;
a chamber attached to the base, the chamber having an internal chamber cavity which is in communication with the internal base cavity;
an access member attached to the chamber, wherein when the access member is disposed in an open position access is provided to the internal base cavity or the internal chamber cavity from outside skin of the living creature;
an inlet port in communication with the internal base cavity or the internal chamber cavity;
an outlet port in communication with the internal base cavity or the internal chamber cavity; and
a transparent viewing member which allows viewing of the internal base cavity or the internal chamber cavity from outside the skin of the living creature.

19. The in-vivo bioreactor system of claim 18 wherein the internal base cavity or the internal chamber cavity contains a biocompatible matrix, a biocompatible scaffold, a decellularized matrix, or a biocompatible engineered system.

20. The in-vivo bioreactor system of claim 18 wherein the internal base cavity or the internal chamber cavity contains a growth factor, a protein, a nutrient, a liquid scaffold, a medication, a treatment, or a cell inserted through the inlet port.

21. The in-vivo bioreactor system of claim 18 wherein a growing tissue or a growing organ is disposed in the internal base cavity or the internal chamber cavity.

22. The in-vivo bioreactor system of claim 18 wherein when the access member is disposed in a closed position, access to the internal base cavity or the internal chamber cavity is closed from outside the skin with the exception of through the inlet and outlet ports.

23. The in-vivo bioreactor system of claim 18 further comprising at least one sensor disposed within or adjacent to the internal base cavity or the internal chamber cavity.

24. The in-vivo bioreactor system of claim 18 further comprising a stimuli member disposed within or adjacent to the internal base cavity or the internal chamber cavity applying stimulation to a growing tissue, or to a growing organ disposed within the internal base cavity or the internal chamber cavity.

25. The in-vivo bioreactor system of claim 18 wherein the in-vivo bioreactor system is made of a biocompatible material.

26. The in-vivo bioreactor system of claim 18 further comprising a plurality of varying chambers which each separately attach and detach from the base for achieving varying functions, each of the varying chambers having an internal chamber cavity which is in communication with the internal base cavity when the chamber is attached to the base.

27. The in-vivo bioreactor system of claim 26 wherein the plurality of varying chambers vary in at least one of size, structure, number or type of inlet or outlet ports, or number or type of sensors.

28. The in-vivo bioreactor system of claim 26 wherein the access member attaches and detaches to each of the plurality of varying chambers.

29. The in-vivo bioreactor system of claim 18 further comprising at least one external system which is configured to deliver a medium to the inlet port, or to monitor or analyze a property of the medium or a material of a tissue or organ after it exits the outlet port.

30. The in-vivo bioreactor system of claim 29 wherein the at least one external system comprises a perfusion system, a temperature sensor, a pH sensor, an oxygen sensor, a flow sensor, a glucose sensor, a protein sensor, or a biological product sensor.

31. A method of using an in-vivo bioreactor system comprising:
locating a base of an in-vivo bioreactor system to be at least partially disposed below a dermis of a living creature;
attaching a chamber of the in-vivo bioreactor system to the base so that an internal chamber cavity of the chamber is in communication with an internal base cavity of the base;
flowing a medium through an inlet port of the in-vivo bioreactor system into the internal base cavity or the internal chamber cavity while the chamber is attached to the base attached to the living creature;
growing a tissue or an organ within the internal base cavity or the internal chamber cavity; and
viewing the growing tissue or the growing organ disposed within the internal base cavity or the internal chamber cavity through a transparent viewing member of the in-vivo bioreactor system.

32. The method of claim 31 further comprising disposing a biocompatible matrix, a biocompatible scaffold, a decellularized matrix, or a biocompatible engineered system within the internal base cavity or the internal chamber cavity with an access member of the in-vivo bioreactor system disposed in an open position.

33. The method of claim 32 further comprising closing the access member locking the biocompatible matrix, the biocompatible scaffold, the decellularized matrix, or the biocompatible engineered system within the internal base cavity or the internal chamber cavity.

34. The method of claim 33 further comprising growing the tissue or the organ within the internal base cavity or the internal chamber cavity from the biocompatible matrix, the biocompatible scaffold, the decellularized matrix, or the biocompatible engineered system.

35. The method of claim 31 further comprising flowing the medium through an outlet port of the in-vivo bioreactor system.

36. The method of claim 31 wherein the medium comprises a growth factor, a protein, a nutrient, a liquid scaffold, a medication, a treatment, or a cell.

37. The method of claim 31 further comprising sensing a property within the internal base cavity or the internal chamber cavity using a sensor of the in-vivo bioreactor system.

38. The method of claim 37 wherein the property comprises a temperature, a pH, oxygen, a flow, a glucose, a protein, or a biological product.

39. The method of claim 31 further comprising applying stimulation to the growing tissue or the growing organ disposed within the internal base cavity or the internal chamber cavity using a stimuli member of the in-vivo bioreactor system.

40. The method of claim 39 wherein the stimulation comprises applying a tension or compression stress or strain to the growing tissue or the growing organ disposed within the internal base cavity or the internal chamber cavity using the stimuli member.

41. The method of claim 31 further comprising using the chamber to achieve a first function for the growing tissue or the growing organ and then detaching the chamber from the base, attaching a varied chamber to the base to dispose an internal chamber cavity of the varied chamber in communication with the internal base cavity, and using the varied chamber attached to the base to achieve a second function for the growing tissue or the growing organ.

42. The method of claim 41 wherein the varied chamber varies from the chamber in at least one of size, structure, number or type of inlet or outlet ports, or number or type of sensors.

43. The method of claim 41 wherein the first and second functions comprise growing or obtaining varying properties, growth levels, growth stages, or results for the growing tissue or the growing organ.

44. The method of claim 41 further comprising detaching an access member from the chamber and then attaching the access member to the varied chamber.

45. The method of claim 31 further comprising delivering the medium to the inlet port through an external system.

46. The method of claim 45 wherein the at least one external system comprises a perfusion system.

47. The method of claim 35 further comprising monitoring or analyzing a property of the medium or material of the tissue or organ after it exits the outlet port using an external system.

48. The method of claim 47 wherein the at least one external system comprises a perfusion system, a temperature sensor, a pH sensor, an oxygen sensor, a flow sensor, a glucose sensor, a protein sensor, or a biological product sensor.
